(19) United States
(12) Patent Application Publication
(10) Pub. No.: US 2018/0250477 A1
(43) Pub. Date: Sep. 6, 2018

(54) NEEDLE FOR INTRALIGAMENTARY ANAESTHESIA

(71) Applicant: Henke-Sass, Wolf GmbH, Tuttlingen (DE)

(72) Inventors: Fabian-Alexander MUELLER, Wurmlingen (DE); Markus MOSER, Villingen-Schwennigen (DE); Lothar TAUBENHEIM, Erkrath (DE)

(21) Appl. No.: 15/913,867

(22) Filed: Mar. 6, 2018

(30) Foreign Application Priority Data
Mar. 6, 2017 (DE) 102017104618.6

(51) Int. Cl. A61M 5/32; A61B 17/34; A61M 5/31

(52) U.S. Cl. CPC A61M 5/3286; A61M 19/00; A61M 5/3134; A61B 17/3472

(57) ABSTRACT

A needle for intraligamentary anaesthesia is provided, wherein the needle has a tubular body with a proximal and a distal end, the body comprises a proximal opening in the proximal end, a distal opening in the distal end and a passage, extending through the body, which connects the two openings, wherein the body is formed in one piece and the outer diameter of the tubular body is at no point greater than 0.30 mm and wherein the distal end narrows in the longitudinal direction from the proximal to the distal end, and is rounded and/or is free from cut edges.

### PRIORITY

This application claims the benefit of German Patent Application No. 102017104618.6, filed on Mar. 6, 2017, which is hereby incorporated herein by reference in its entirety.

### FIELD

The present invention relates to needles for intraligamentary anesthesia, as well as syringes with such a needle.

### BACKGROUND

In the case of intraligamentary anesthesia, the needle is inserted into the periodontal space or the desmodontal space (space between tooth cervix and gingival margin) until there is contact with bone. The anaesthetic is then injected into the periodontal space of the corresponding tooth root, with the result that the periodontium, including the bony alveolus up to the root tip of the tooth, is permeated with the anaesthetic. The nerve fibres reaching into the dental pulp are thereby numbed within a few seconds. In the case of intraligamentary anaesthesia, advantageously, less anaesthetic per tooth is administered, which can be particularly advantageous for patients at risk of a cardiovascular event. As the needle is ground so as to be pointed, the pain from the injection prick can be kept relatively low. Irrespective thereof, however, there is always some pain from the injection prick, as tissue is damaged irrespective of how deep into the desmodontal space the needle is inserted. This is disadvantageous precisely in the case of patients with a fear of needles, as each tooth is to be numbed individually.

### SUMMARY

An object of certain embodiments of the invention is to provide an improved needle for intraligamentary anaesthesia.

The distal end of the needle narrows in the longitudinal direction from the proximal to the distal end, and is rounded and/or is free from cut edges. Thus, it is possible to insert the needle into the periodontal space without damaging tissue. This insertion can be carried out until there is contact with bone. The risk of damage can thus be minimized and there is no pain from the injection prick.

In particular, the distal end can be spherically rounded. It is preferred here for the radius of the spherical rounding to amount to half the outer diameter of the tubular body of the needle.

The distal end can be ground such that it does not have a grinding angle. Furthermore the distal end can be ground such that it is semi-circular,

The outer diameter of the needle can be 0.30 mm or less. Outer diameters of 0.300 mm and 0.28 mm are preferred. The inner diameter can be 0.133 mm, 0.165 mm or also 0.140 mm. Thus there are then wall thicknesses of 0.0835 mm, 0.0675 mm or 0.08 mm with an outer diameter of 0.3 mm and wall thicknesses of 0.0735 mm, 0.0575 mm and 0.07 mm with an outer diameter of 0.28 mm.

The passage preferably extends coaxially to the longitudinal axis.

The tubular body preferably has an annular outer contour in cross section, with the result that the outer diameter is clearly defined. In the case of a different outer contour in cross section, by outer diameter is meant the diameter of the smallest circular ring in which the outer contour of the tubular body is completely contained.

The tubular body preferably has the same outer contour in cross section over its entire length. In particular, the tubular body is formed as a hollow cylinder (in each case preferably with the exception of the proximal and distal ends). In particular, the tubular body has a constant outer diameter over its entire length.

The tubular body (with the distal end or optionally without the proximal end) is preferably rotationally symmetrical relative to the longitudinal axis. The tubular body can thus be mapped onto itself by rotation through a particular angle. Thus, the tubular body in cross section can in principle have the outer contour of a triangular, a rectangular or another rotationally symmetrical polygonal area. However, the outer contour of a circular cross-sectional shape is preferred. In particular, the tubular body can be rotationally symmetrical relative to the longitudinal axis. In this case, the cross section is annular.

The tubular body thus, preferably together with its distal end, has rotational symmetry or symmetry of rotation (mapping onto itself at any angles of rotation about the longitudinal axis). The proximal end can preferably be ground so as to be pointed and/or such that it has at least one cutting edge, with the result that the proximal end is not rotationally symmetrical.

The needle can furthermore have a socket. The socket can serve, e.g., to connect the needle to a syringe (e.g. a cartridge syringe). The socket can be glued or welded to the tubular body. Furthermore, the socket can be rotationally symmetrical or symmetrical in rotation.

The distal end can be formed such that the radius of the rounding increases in the direction towards the distal end.

The needle or the tubular body can extend in a straight line. It is furthermore possible for the needle or the tubular body to be curved.

A syringe with a needle is also disclosed. The syringe can be formed as a cartridge syringe or carpule syringe.

An anaesthesia method is furthermore disclosed, in which a syringe with a needle according to the invention is pushed into the periodontal space, wherein the distal end of the needle points away from the syringe. In the pushed-in state, the anaesthetic is then applied through the needle and via the distal end.

The method can have further steps, which are described in connection with the needle according to the invention and the syringe with the needle according to the invention.

It is understood that the features mentioned above and those yet to be explained in the following are applicable, not only in the specified combinations, but also in other combinations or singly, without departing from the scope of the present invention.

### BRIEF DESCRIPTION OF THE DRAWINGS

FIG. 1 is a schematic representation of an embodiment of the needle in accordance with embodiments of the invention.

FIG. 2 is a magnified cross-sectional view of the distal and proximal ends of the tubular body of the needle of FIG. 1.

FIG. 3 is a perspective representation of a syringe.

FIG. 4 is a sectional view of the syringe of FIG. 3.

FIG. 5 is a schematic representation of a cartridge.

FIG. 6 is the syringe according to FIGS. 3 and 4 with the cartridge according to FIG. 5 and the needle according to FIGS. 1 and 2.

FIG. 7 is a schematic representation of a tooth.

FIG. 8 is a schematic representation of the insertion of the needle connected to the syringe into the periodontal space.

FIG. 9 is a sectional view of the distal end of a further embodiment of the needle in accordance with embodiments of the invention.

FIG. 10 is a sectional view of the distal end of a further embodiment of the needle in accordance with embodiments of the invention.

While the invention is amenable to various modifications and alternative forms, specifics thereof have been shown by way of example in the drawings and will be described in detail. It should be understood, however, that the intention is not to limit the invention to the particular example embodiments described. On the contrary, the invention is to cover all modifications, equivalents, and alternatives falling within the scope of the invention as defined by the appended claims.

### DETAILED DESCRIPTION

The invention is explained in yet more detail below with the aid of embodiment examples with reference to the attached drawings, which also disclose features essential to the invention. These embodiment examples merely serve the purpose of illustration and are not to be interpreted as limiting. For example, a description of an embodiment example with a plurality of elements or components is not to be interpreted to the effect that all of these elements or components are necessary for the implementation. Rather, other embodiment examples can also contain alternative elements and components, fewer elements or components or additional elements or components. Elements or components of different embodiment examples can be combined with each other, unless otherwise indicated. Modifications and alterations which are described for one of the embodiment examples can also be applicable to other embodiment examples. To avoid repetitions, the same or corresponding elements are given the same reference numbers in different figures and are not explained repeatedly.

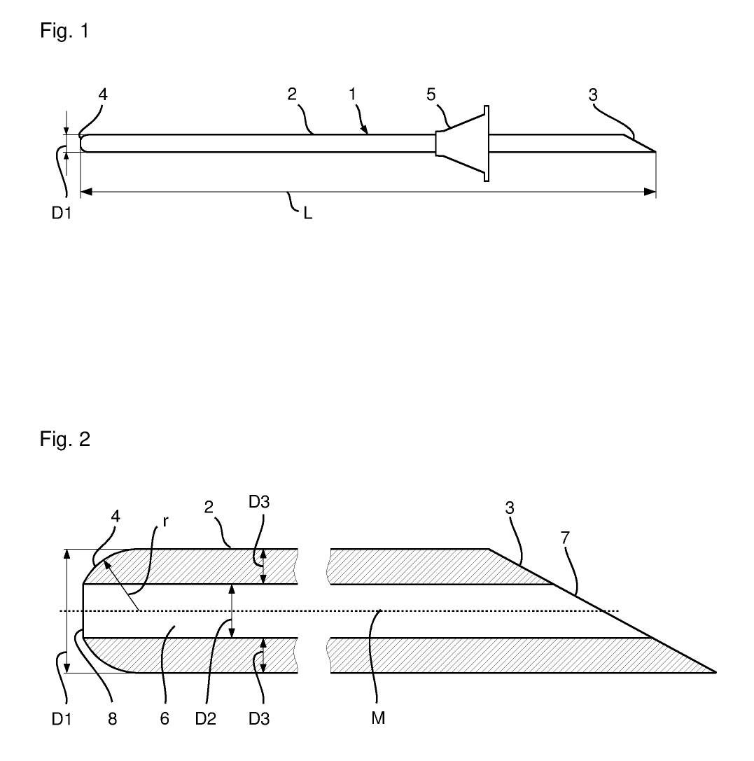

In the embodiment shown in FIG. 1, the needle 1 according to the invention comprises a tubular body 2, which has a proximal end 3 and a distal end 4, and a socket 5. The needle 1 according to the invention can in particular be used in intraligamentary anaesthesia, in which an anaesthetic is applied via the needle 1, wherein the needle 1 can be pushed, for example, into the periodontal space at the root of the corresponding tooth.

In order to prevent tissue damage as far as possible, on the one hand the needle 1 according to the invention is formed particularly thin and on the other hand the distal end 4 is rounded in the described embodiment. In addition, the tubular body 2 is formed in one piece.

As can be seen in particular from the partial sectional view of the proximal and distal ends 3, 4 in FIG. 2, the body 2 has a passage 6 (which can also be called a bore 6), which extends from a proximal opening 7 at the proximal end of the body 2 up to a distal opening 8 at the distal end 4 of the body 2. The tubular body 2 is rotationally symmetrical relative to a central axis M, which extends from the proximal to the distal end 3, 4, and has a circular outer contour in a section plane perpendicular to the plane of drawing in FIG. 2. Furthermore, the passage 6 has a circular cross section in this section plane and extends coaxially to the central axis M.

The tubular body 2 has an outer diameter D1 of less than or equal to 0.30 mm. In the embodiment described here the outer diameter D1 is 0.30 mm. The inner diameter D2 of the tubular body 2, and thus the diameter of the passage 6, is 0.133 mm, with the result that the tubular body 2, which can also be called a hollow needle, has a wall thickness D3 of 0.0835 mm.

The proximal end 3 of the body 2 is ground, as is represented in FIG. 2, in order to be able to penetrate a membrane of a cartridge with the proximal end 3, as will be described below.

The distal end 4 is rounded. In the embodiment described here, the distal end is spherically ground, wherein the radius r of the ground section preferably amounts to half the outer diameter D1, and thus is 0.15 mm here.

The tubular body 2 can be produced from steel or stainless steel. Because of the one-piece formation and the continuous wall, despite the small outer diameter, there is the necessary stability, even in the case of a length L of the body 2 of, e.g., from 9 to 15 mm. The body 2 is in particular characterized in that it has no further openings apart from the two openings 7 and 8. In particular, there are thus no lateral outlet openings in the wall, with the result that the body 2 is free from lateral outlet openings or inlet openings. In particular, the tubular body 2 has a constant outer diameter over its entire length, of course with the exception of the rounded distal end 4, which narrows, and the proximal end 3, which is ground so as to be pointed. It is furthermore preferred for the diameter of the passage 6 to be constant over its entire length, with the result that the thickness of the wall of the body 2 is also constant.

The socket 5 can consist of plastic or metal and is, e.g., glued or welded to the tubular body 2.

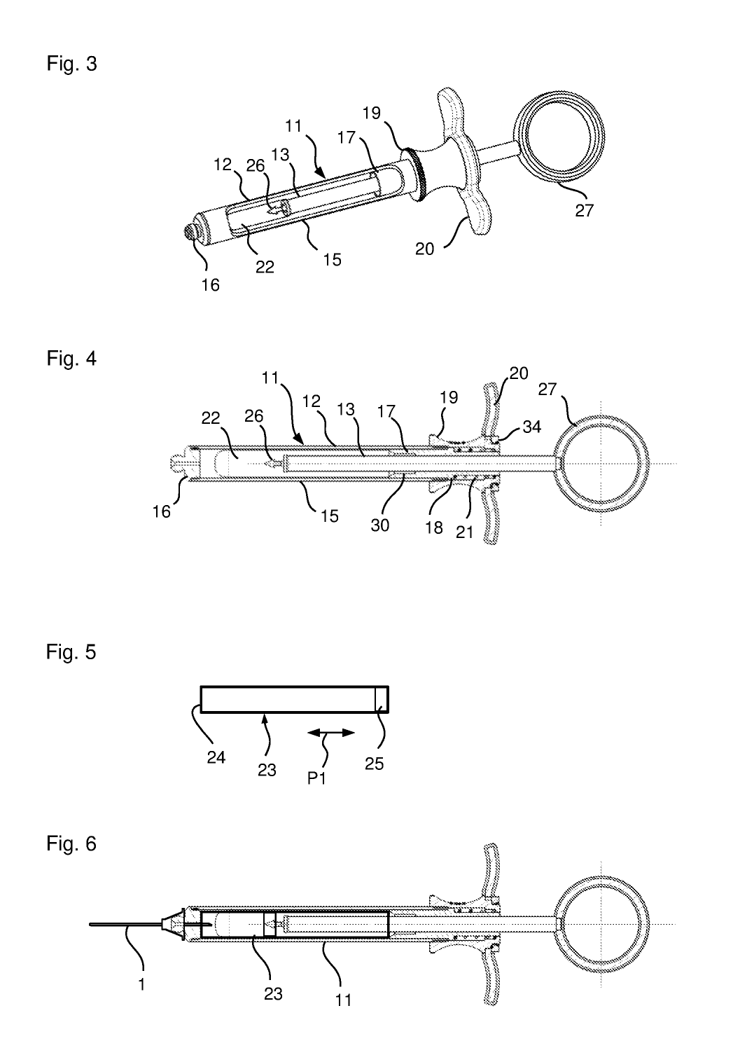

A syringe 11 which can be used together with the needle 1 according to the invention is shown in FIGS. 3 and 4. The syringe 11 can, e.g., be formed as a cartridge syringe, which can also be called a carpule syringe. The syringe 11 comprises a syringe body 12 formed substantially tubular, in which a plunger 13 is held so that it can be shifted longitudinally.

The syringe body 12 comprises a main tube 15 in the shape of a hollow cylinder with a needle receiver 16 at the front end and a centering sleeve 17 installed at the rear end. The needle receiver 16 is screwed and glued into the main tube 15. The centering sleeve 17 is inserted into the main tube 15 up to its stop 18, which rests against the rear end of the main tube 15. Furthermore, at the rear end of the main tube 15, a handle cap 19 is screwed on and glued, on the rear end of which a finger plate 20 is rotatably mounted and secured by a ring 34. For this, the ring 34 is screwed and glued onto the rear end of the finger plate 20. Between the centering sleeve 17 and the handle cap 19, a coil spring 21 is arranged, the ends of which are connected to the centering sleeve 17 and the handle cap 19, with the result that the centering sleeve 17 is pressed against the main tube 15.

The main tube 15 has a lateral loading opening 22, via which the syringe 11 can be loaded with a cartridge 23 shown schematically in FIG. 5. The cartridge 23 can be a conventional glass ampoule, which is sealed at its front end with a membrane layer 24 and at its rear end with a rubber stopper 25 that can be shifted in the longitudinal direction of the cartridge 23 (as is indicated by the double arrow P1).

At its front end, the plunger 13 of the syringe 11 has an arrowhead 26, which can be pushed into the rubber stopper 25 of the inserted cartridge 23 such that the rubber stopper 25 can be shifted in the longitudinal direction by the movement of the plunger 13. The arrowhead 26 can thus be anchored in the rubber stopper 25.

At the rear end of the plunger 13, an annular thumb ring 27 is secured for the thumb of a person using the syringe 11. The previously described parts of the syringe 11 are all produced from metal, wherein the plunger 13 consists of stainless steel and the remaining parts (except for the compression spring 21) are produced from chrome-plated brass. The syringe body 12 has a length of approx. 110 mm and the inner diameter of the main tube is approx. 10 mm. The plunger 13 has a length of approx. 100 mm. The outer and inner diameters of the thumb ring 27 are approx. 32 or 30 mm.

In operation, the plunger 13 is pulled backwards by means of the thumb section 27 until the arrowhead 26 is completely sunk in a receiver area 30 at the front end of the centering sleeve 17. A further pulling-back of the plunger 13 leads to the centering sleeve 17 being shifted in the longitudinal direction of the main tube 15 against the spring 21, with the result that there is enough space in the main tube 15 to insert the cartridge 23. The plunger 13 is held in this position while the cartridge 23 is being inserted. As soon as the cartridge 23 has been inserted into the main tube 15 via the lateral loading opening 22, the holding of the position of the plunger 13 can be ended, with the result that, because of the restoring force of the spring 21, the centering sleeve 17 presses the inserted cartridge 23 against the needle receiver 16 and thus fixes the cartridge 23 in the main tube 15.

The plunger 13 is then pushed forwards in order to stab the arrowhead 26 into the rubber stopper 25. Furthermore, the needle 1 is stabbed through the membrane layer 24 of the cartridge 23 through the needle receiver 16 and the membrane 24. This is easily possible because of the ground or sharpened proximal end 3 of the needle 1. The syringe 11 is thereby prepared for use.

In the position shown in FIG. 6, the rubber stopper 26 is already pushed beyond halfway to the front end of the syringe 1, with the result that the corresponding fluid, which was present in the cartridge 23, has been dispensed via the needle 1 and thus via the distal end 4. The fluid can be, for example, an anaesthetic.

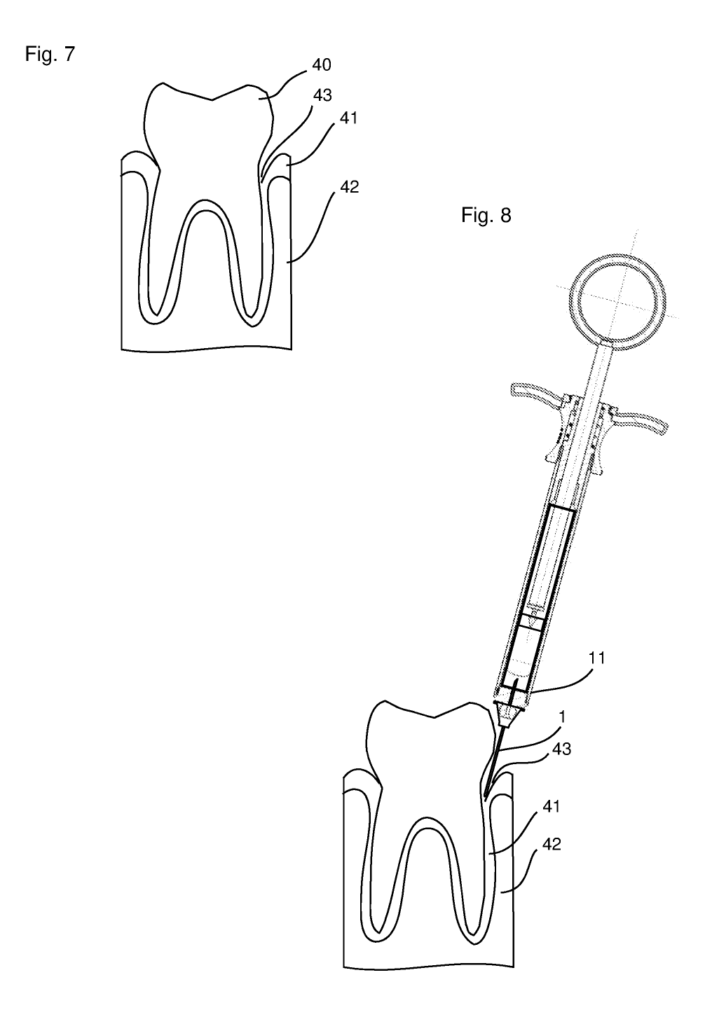

A tooth 40, together with the gum 41 and the corresponding bone 42 (e.g. alveolar bone), is represented in a schematic view in FIG. 7. The periodontal space 43 lies between the gum 41 and the tooth 40.

In order now to numb this tooth 40 and the area surrounding the tooth 40, the syringe 11 is positioned such that the proximal end 3, which is rounded, is inserted into the periodontal space 43. This can be effected, for example, such that there is contact with bone. The needle 1 is thus inserted into the ligaments of the periodontium, the Sharpey's fibres. The anaesthetic can then be injected there via the needle 1. The anaesthetic can penetrate the periodontium, including the bony alveolus up to the root tip of the tooth, and numbs the nerve fibres reaching into the dental pulp there within a few seconds.

As the distal end 4 is rounded, the tissue damage that has always occurred until now can be prevented in the case of needles with sharpened distal end. Damage can thus be prevented and there is also no pain from the injection prick.

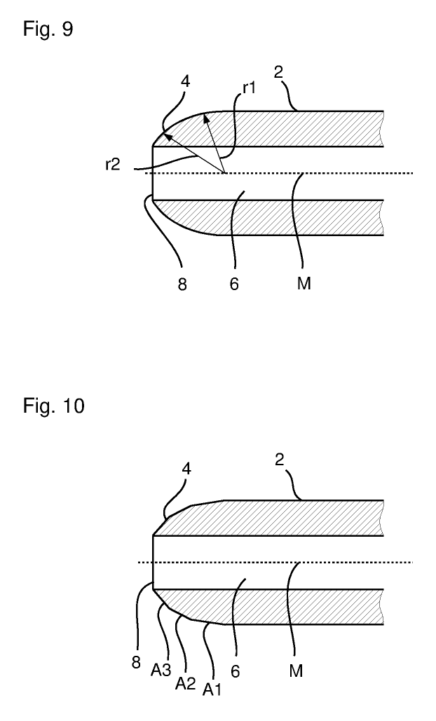

The distal end 4 of the needle 1 need not be spherically ground. It is important that the distal end 4 narrows in a direction along the central axis from the proximal end 3 to the distal end 4, and is free from cut edges and/or is rounded. In the embodiment shown in FIG. 9 the radius of curvature increases in this direction, with the result that the radius of curvature r1 is smaller than the radius of curvature r2. Of course, the radius of curvature can also decrease in this direction.

Furthermore, it is possible for the rounded shape to be approximated by one or more sections A1-A3, as is represented schematically in FIG. 10. The sections A1-A3 can be, for example, segments of the surface of a cone.

While the invention has been described in connection with what is presently considered to be the most practical and preferred embodiments, it will be apparent to those of ordinary skill in the art that the invention is not to be limited to the disclosed embodiments. It will be readily apparent to those of ordinary skill in the art that many modifications and equivalent arrangements can be made thereof without departing from the spirit and scope of the present disclosure, such scope to be accorded the broadest interpretation of the appended claims so as to encompass all equivalent structures and products. Moreover, features or aspects of various example embodiments may be mixed and matched (even if such combination is not explicitly described herein) without departing from the scope of the invention.

What is claimed is:

1. A needle for intraligamentary anaesthesia, comprising:
a tubular body with a proximal and a distal end, the body including a proximal opening in the proximal end, a distal opening in the distal end and a passage, extending through the body, which connects the proximal and distal openings,
wherein the body is formed in one piece and an outer diameter of the tubular body is at no point greater than 0.30 mm, and
wherein the distal end narrows in the longitudinal direction from the proximal to the distal end, and is at lease one of rounded and free from cut edges.

2. The needle of claim 1, wherein the distal end is spherically rounded.

3. The needle of claim 2, wherein the distal end is rounded with a radius which amounts to half the outer diameter of the tubular body.

4. The needle of claim 2, wherein the tubular body has a constant outer diameter over its entire length.

5. The needle of claim 2, wherein the tubular body has no further openings in addition to the distal and the proximal openings.

6. The needle of claim 1, wherein the distal end is rounded with a radius of curvature which increases in the direction towards the distal end.

7. The needle of claim 6, wherein the tubular body has a constant outer diameter over its entire length.

8. The needle of claim 6, wherein the tubular body has no further openings in addition to the distal and the proximal openings.

9. The needle of claim 1, wherein the tubular body is rotationally symmetrical relative to a longitudinal axis of the tubular body or symmetrical in rotation relative to the longitudinal axis.

10. The needle of claim 1, wherein the proximal end is sharpened.

11. The needle of claim 1, wherein the tubular body has a constant outer diameter over its entire length.

12. Needle according to claim 11, wherein the tubular body has no further openings in addition to the distal and the proximal openings.

13. The needle of claim 1, wherein the tubular body has no further openings in addition to the distal and the proximal openings.

14. The needle of claim 1, wherein the tubular body consists of steel.

15. A syringe comprising a needle according to claim 1.

16. The syringe of claim 15, wherein the syringe is a cartridge syringe.

17. An anaesthesia method for numbing a tooth, comprising:
pushing a syringe with a needle according to claim 1 into the periodontal space between the gum and the tooth, thereby defining a pushed-in state, wherein the distal end of the needle points away from the syringe; and
applying the anaesthetic through the needle and via the distal end when the needle is in the pushed-in state.
